(19) United States
(12) Patent Application Publication
(10) Pub. No.: US 2008/0139661 A1
(43) Pub. Date: Jun. 12, 2008

(54) Fragranted moistening fluids that destroy and/or inhibit the growth of biological organisms while minimizing a tacky build up

(75) Inventors: Richard A. Bernard, Norwalk, CT (US); William E. Ryan, Monroe, CT (US)

Correspondence Address: PITNEY BOWES INC.;35 WATERVIEW DRIVE, P.O. BOX 3000, MSC 26-22, SHELTON, CT, 06484-8000, US

(73) Assignee: Pitney Bowes Incorporated, Stamford, CT (US)

(21) Appl. No.: 11/599,900

(22) Filed: Nov. 15, 2006

(51) Int. Cl. A01N 33/12; A01N 25/00; A01P 1/00; A01P 3/00

(52) U.S. Cl. 514/643; 514/772

(57) ABSTRACT

Moistening fluids are disclosed that are capable of being used in mail processing machines and systems. Mail processing equipment can automatically feed and moisten envelopes at slow to very high speeds of 30 inches per second. The moistening system becomes contaminated with paper dust, talc, and common envelope adhesives. The moistening fluids kill many types of bacteria, fungi, and inhibit the growth of other types of bacteria, fungi, and algae. The moistening fluids of this invention are safe for use in an office environment, i.e., they are non-toxic, not tacky, have a pleasant odor, are non-flammable, have no residue build up and may be transported by common carriers without any safety precautions. The moistening fluid contains: detergent, biocide, alcohol, dye, de-ionized water and a fragrance.

### CROSS REFERENCE TO RELATED APPLICATIONS

Reference is made to commonly assigned co-pending patent application Ser. No. 11/011,268 filed Dec. 14, 2004, entitled “MOISTENING FLUIDS THAT DESTROY AND/OR INHIBIT THE GROWTH OF BIOLOGICAL ORGANISMS” in the names of Maureen A. Spisinski, Richard A. Bernard and William E. Ryan, Jr. and Ser. No. 11/300,995 Filed Dec. 15, 2005, entitled “Fragranted MOISTENING FLUIDS THAT DESTROY AND/OR INHIBIT THE GROWTH OF BIOLOGICAL ORGANISMS” in the names of Richard A. Bernard and William E. Ryan, Jr.

### FIELD OF THE INVENTION

The invention relates generally to moistening fluids and more particularly to moistening fluids that destroys and/or inhibit the growth of biological organisms.

### BACKGROUND OF THE INVENTION

In mail processing systems, moistening devices are used to wet the flap of an envelope in preparation for sealing the envelope or moistening a tape for sealing objects, or adhering labels. Conventionally, flap wetting is accomplished by feeding the envelopes flaps past a moisture applicator such as a brush, a wick, or other moist surface, allowing the moist surface to come into contact with the water-moistening adhesive on the envelope flap. The prior art also utilized sprayer systems to moisten envelope flaps. The moisture causes the adhesive to soften and to become sticky. The moistened envelopes' flaps are closed and the adhesive is pressed between the flap and the body of the envelope to form a seal. The envelope is then either ejected into a stacker, or passed on to another part of the mail processing system for further processing.

Envelope flap moistening devices generally fall into two categories, contact and non-contact moistening devices. Contact systems generally deposit moisture onto an envelope flap by contact with a wetted substrate or narrow slotted device that allows fluid to flow, based upon the capillary action of the slotted device's contact with the flap. Non-contact systems generally spray moisture onto the envelope flap. In non-contact flap moistening systems, envelope flap moistening has been performed with a nozzle and the aid of a pumping system. In the aforementioned systems the moistening fluid is stored in a reservoir or remains in internal tubing, where certain types of bacteria, fungi, and algae have an opportunity to grow.

The bacteria, fungi and algae have a natural source of food because the envelope adhesive generally contains dextrin i.e., a corn and/or potato starch.

The following microorganisms were found in representative samples taken from moistening systems: Sphingomonas paucimobilis (bacteria); Geotrichum species (fungus); Yeasts; Pseudomonas stutzeri (bacteria); Fusarium species (mold); Aspergillus niger (mold); Acinetobacter species (bacteria); Blue green algae; Caulobacter species (bacteria); Pseudomonas aeruginosa (bacteria); Pseudomonas fluorescens (bacteria); Brevendimonas species (bacteria); Flavomonas species (bacteria); Cladosporium species (fungus); Oididendron species (fungus) Penicillium species (mold).

A disadvantage of current moistening devices is that they use moistening fluids that are conducive to the growth of bacteria, fungi, and algae.

Another disadvantage of current contact moistening devices is that sometimes-significant amounts of bacteria, fungi, and algae grow before the moistening fluid is completely used, and due to the inappropriate selection of a biocide or an insufficient concentration of the biocide, it is not adequate to stop growth.

A further disadvantage of current contact moistening devices is that the growth of bacteria, fungi, and algae may result in the inconsistent wicking of the substrate causing it to become unevenly saturated, which results in decreased moistening and/or clogging of the filters and tubing in moistening systems.

A further disadvantage of current systems is that the excessive growth of bacteria, fungi, and algae results in the production of unpleasant odors.

A further disadvantage of some current moisteners is that they have an alcohol type odor.

An additional disadvantage of some current moisteners is that the evaporation of the moistening fluid causes a tacky residue build up.

A still further disadvantage of some current moisteners is that the evaporation of the moistening fluid causes envelope feeding deck surfaces to become tacky and lead to misfeeding of mail pieces.

### SUMMARY OF THE INVENTION

This invention overcomes the disadvantages of the prior art by providing moistening fluids that destroys many types of bacteria, fungi, and inhibits the growth of other types of bacteria, fungi, and algae. The moistening fluids of this invention are safe for use in an office environment, i.e., they are non-toxic, have no tacky residue build up and may be transported by common carriers without any safety precautions. The moistening fluids may be used in mailing systems to seal envelope flaps, adhere labels to mail pieces, i.e., letters, flats, or packages. They may also be used to adhere labels and tapes to objects. The moistening fluids may also be placed in a device or dispenser that is a stand alone container.

An advantage of this invention is that the surface tension of the moistening fluid is within the range of 27.1-40.6 dynes/cm. Disinfectants are usually solutions of low surface tension. This allows them to spread out on the cell walls of bacteria and disrupt them.

Properties of the moistening fluids under consideration are conductivity, alkalinity/acidity, wicking, surface tension, plastic compatibility, moistening sealing weight, viscosity, zone of inhibition, turbidity, specific gravity, rolling tack and challenge test.

Conductivity is the ability of a material to conduct electric current. Since the charge on ions in solution facilitates the conductance of electrical current, the conductivity of a solution is proportional to its ion concentration. Thus, the conductivity is an important physical parameter in the preparation of the moistening fluids described herein.

pH is a value taken to represent the acidity or alkalinity of an aqueous solution; it is defined as the logarithm of the reciprocal of hydrogen-ion concentration of a solution.

Alkalinity is the measurement of pH value above 7 and acidity is the measurement of a pH value below 7.

The pH value is important, because it demonstrates the moistening fluid's ability to be handled safely and operate safely in most systems.

The ability to destroy and/or inhibit the growth of certain types of bacteria, fungi, and algae is improved by the addition of detergents, biocides, and alcohols at specific ratios.

Rolling tack is a measure of the resistance of a cylinder to roll down an incline and a measure of time it takes. A coated film is allowed to evaporate and placed on the incline prior to placing the cylinder in place.

A further advantage of this moistening fluid is that it has a pleasant odor.

An additional advantage of this moistener is that it has a pleasant odor masking out the alcohol smell.

An additional advantage of this invention is that the moistening fluid prevents a sticky or tacky residue build up upon evaporation of the moistening fluid.

An additional advantage of this invention is that the moistening fluid prevents a sticky or tacky residue build up in the wicks and/or applicators of moistening systems upon evaporation of the moistening fluid.

An additional advantage of this invention is that the moistening fluid prevents a sticky or tacky residue build up in the reservoirs of moistening systems upon evaporation of the moistening fluid.

An additional advantage is that the moistening fluid of this invention residue build upon evaporation does not cause a tacky surface on the feeding deck of a postage meter unit which will not cause non-feeding or skewing of envelopes.

### DETAILED DESCRIPTION OF THE PREFERRED EMBODIMENT

The formulation of the moistening fluids of this invention is capable of being used in mail processing machines and systems. Composition of the moistening fluids, in accordance with the invention comprises detergent, biocide, alcohol, dye, fragrance and water.

The general composition of the moistening fluids is as follows:

| INGREDIENT | (WT. %) |
|---|---|
| Detergent | Range .25–5.0 |
| Biocide | 0.023 |
| Alcohol | 4.95 |
| Dye | 0.000015 |
| Fragrance | .035 |
| De-ionized Water | Range 89.99199–94.74199 |

The following detergents were used in the examples described herein:
1. Burco HCS—50 NF manufactured by Burlington Chemical of Post Office Box 111, 615 Huffman Mill Road, Burlington, N.C.
2. X-20146 manufactured by Kao Specialties Americas LLC, 243 Woodbine Street, P.O. Box 2316, High Point, N.C. 27261.
3. Burco LAF—3420 manufactured by Burlington Chemical of Post Office Box 111, 615 Huffman Mill Road, Burlington, N.C.
4. Burco LAF—345 PB manufactured by Burlington Chemical of Post Office Box 111, 615 Huffman Mill Road, Burlington, N.C.

The following biocide was used in the examples described herein:
1. Alkyl(C₁₄ 50%, C₁₆ 10%, C₁₂ 40%) Dimethyl Benzyl Ammonium Chloride manufactured by Lonza of 17-17 Route 208, Fair Lawn, N.J.

The following Alcohol was used in the examples described herein:
1. 2-Propanol

The following Dye was used in the examples described herein:
1. FD &C #1 Blue Dye—(Food Drug & Cosmetic #1 Blue Dye) manufactured by Pylem Products Company Inc. 2175 East Cedar Street, Tempe, Ariz. 85281-7431.

The following waters were used in the examples described herein:
1. De-ionized water
2. Distilled water
3. Tap water

The following Fragrances were used in the examples described herein:
1. Citrus 60001578 manufactured by Bell Flavors And Fragrance Inc. 500 Academy Drive, Northbrook, Ill. 60062.
2. French Vanilla A44676 (25% Scent) manufactured by Atlantis Aromatics, Inc. 5047 Industrial Road, Suite 4, Wall, N.J. 07719.
3. Pine Tree A44680 (25% Scent) manufactured by Atlantis Aromatics, Inc. 5047 Industrial Road, Wall, N.J. 07719.
4. Peppermint A44681 manufactured by Atlantis Aromatics Inc. 5047 Industrial Road, Suite 4, Farmington, N.J. 07727.

To determine the efficacy of the moistening fluids to inhibit and/or kill bacteria, fungi, and algae, a Zone of Inhibition test was conducted. The procedure for Zone of Inhibition testing is as follows:

Equipment
Sterile Tryptic Soy Agar plates (TSA)
Sample contaminants in sterile diluent solution manufactured by Fabriqué au Canada par Starplex Scientific Inc., 50 Steinway Blvd., Etobicoke, Ontario, Canada
The sample contaminant used contained one or more of the following organisms:
   1. Geotrichum species (fungus)
   2. Yeasts
   3. Pseudomonas stutzeri (bacteria)
   4. Fusarium species (mold)
   5. Aspergillus niger (mold)
   6. Acinetobacter species (bacteria)
   7. Blue green algae
   8. Caulobacter species (bacteria)
   9. Pseudomonas aeruginosa (bacteria)
   10. Pseudomonas fluorescens (bacteria)
   11. Brevendimonas species (bacteria)
   12. Flavomonas species (bacteria)
   13. Cladosporium species (fungus)
   14. Oididendron species (fungus)
   15. Sphingomonas paucimobilis (bacteria)
   16. Penicillium species (mold)
16 mm discs cut from Whatman 41 filter paper
Millipore 5.0 um 25 mm discs
Pall 45 um membrane filters
Metric ruler
Moistening fluid (Examples 1-21)
Distilled water
Incubating oven set for 35° C.

Procedure
Swab sterile Tryptic Soy Agar plates (TSA) with solution of sample contaminant.
Place one disc, or membrane filter, which has been saturated with the moistening fluid on center of plate.
Saturate one disc in distilled water and utilize as a control
Incubate overnight in 35° C. incubating oven.
At 24-hour intervals, measure the linear distance that has been cleared of microbial growth and record from the filter disc's circumference to the outer perimeter of the cleared area. This is the Zone of inhibition.
Take measurements each 24-hour period for three (3) days.

To determine the ability of the moistening fluids to seal envelopes in mailing systems envelope, sealing tests were performed.

Equipment:
#10 envelopes with water moisturizing adhesive
Moistening fluids, as described
Mettler PE 3600 balance
Moistening Mailing system(s)
Deionized and/or Distilled water

Procedure:
One hundred milliliters (mls) of moistening fluid to be tested was added to the moistening fluid reservoir of the moistening mailing system.
Fifty (50) #10 envelopes with moisturizing adhesive were weighed dry.
These same fifty (50) envelopes were then sent through the moistening mailing system, in order to wet and seal the envelopes.
The 50 envelopes were re-weighed immediately to determine the amount of gross weight gain of the envelopes.
The mailing system was rinsed with either distilled or deionized water, in order to remove traces of the previously run moistening fluid. This was accomplished by running twenty-five (25) envelopes through the system with either distilled or deionized water.
This procedure was conducted a minimum of two times, for each example in order to determine the average weight gain of fifty (50) envelopes of each moistening fluid.
A baseline average weight gain was determined by averaging the gross weight gain for each group of fifty (50) envelopes tested, for each example.

To determine the speed of wicking, i.e., the ability of a fluid to be drawn up the fibers, of a brush of a moistening system, wicking tests were performed.

Equipment:
Esterlon (polyester) F984031, unbaked brushes, flagged 1 pass 1.5 seconds/side
Ring stand equipped with two side arm clamps
Lap/split timer by Fisher Scientific of Hanover Park, Ill.
Whatman 11.0 cm 40 Ashless filter paper circles
Moistening fluids as described
Sterile empty petri dishes
Procedure: Small ring stand with two side arm clamps was assembled in hood
One side arm clamp was used to hold the Esterlon brush in an upright position
The second side arm clamp held the filter paper circle fixed on the brush's fibrous end
Sterile petri dish was filled with the appropriately labeled moistening fluids
At the point where the lower end of the brush came in contact with the moistening fluids, the timer was activated to measure the time it took the moistening fluids to travel up the bristles of the brush and moisten the filter paper.
Once moisture was visible on the filter paper, the timer was stopped and the time recorded.

To determine the ability of the moistening fluids to inhibit and/or destroy the growth of specific bacteria, fungi and algae, in mailing systems, the moistening fluid was subjected to Challenge Testing (Modified American Society For Testing Materials (ASTM) D-2574), with the following organisms:

| | |
|---|---|
| Acinetobacter sp. | Sacchromyces cereviseae |
| Penicillium sp. | Candida albicans |
| Cladosporium sp. | Pseudomonas aeruginosa |
| Geotrichum sp. | Escherichia coli |
| Caulobacter sp. | Aspergillus niger |
| Mixed Algae Pond Collection | Controls of Distilled Water and |
| | Tap Water |

The Challenge Test consists of a 7-day study for the above mentioned microorganisms in which each microorganism is inoculated into an aliquot of moistening fluid and subsequently tested as to the viability of each organism, after specific increments of contact time.

Material:
Five (5) test tubes with 9.0 ml. of sterile diluent solution for each microorganism tested; American Type Culture Collection (ATCC) cultures of specified type;
Thirty-two (32) Sterile Tryptic Soy Agar plates, sterile disposable 1 ml. pipettes; pipette aids;

Procedure:

### Obtain pure stock culture of each organism from accredited vendor, such as American Type Culture Collection (ATCC). Perform serial dilutions to determine actual inoculum microbial count for each organism.

Serial dilutions performed by
   Take five test tubes for each organism, each containing 9.0 ml. of sterile diluent solution, and label with appropriate dilution factor (i.e. 1:10; 1:100, 1:1000, 1:10,000 etc.)
   Take 1 ml. of the original inoculum and inoculate the first tube of 9.0 ml. of sterile diluent solution.
   Mix well and plate 0.1 ml. of the diluent solution/microbial culture to each of two (2) sterile Tryptic Soy Agar plates.
   Draw a 1 ml. aliquot from the first dilution (1:10) and inoculate the second dilution (1:100)
   Mix well and plate one (1) ml. of the dilution to each of two (2) sterile Tryptic Soy Agar plates.
   Continue to transfer the same amount (1 ml.) to each successive dilution and mix well.
   Continue to plate 1 ml. of each dilution to two (2) sterile Tryptic Soy Agar plates.
   Incubate at optimal temperature for each organism. After 48 hours, count the colony forming units (CFU) on each plate, at each dilution and record. The number of microorganisms in the original inoculum equals the averaged number of colony forming units (cfus) from the duplicate plates times the dilution of the sample.
Once the number of bacteria/ml for each microorganism being tested has been determined:
   Prepare a test tube for each tested microorganism with 9.9 mls. of the moistening fluid.
   Inoculate 0.1 mls. of the microorganism to be tested into the 9.9 mls. of moistening fluid and mix well.
   Immediately draw one ml. (1 ml.) of the freshly inoculated moistening fluid and plate onto a sterile Tryptic Soy Agar plate. Continue by plating the duplicate plate.
   Swirl plate to ensure the even distribution of the fluid. Mark the duplicate plates with the organism's identification and designate as Time 0.
Allow the vials with the moistening fluid/microorganism inoculum to remain undisturbed in a biological safety cabinet for 24 hours.
   At the 24 hour mark, mix each vial well and draw one (1) ml. of the moistening fluid/microorganism inoculum and plate onto a sterile Tryptic Soy Agar plate. Plate duplicate plate. Label as Time 24
   Repeat the procedure at the 48 hour and 72 hour period of contact, and label as Time 48 and Time 72, respectively.
   Incubate all of the plates at 27° C. for seven days.
   Read the countable plates (those plates with cfus between 30-300)
   Record the number of cfus/ml for each plate.
Determine the log reduction achieved within 24 hours of contact time, 48 hours of contact time and 72 hours and 7 days of contact time, for each microorganism tested.

Log Reduction Explanation:

“Log” stands for logarithm, which is the exponent of 10. For example, log2 or 10×10 or 100 for a 10-fold or one decimal or 90% reduction in numbers of recoverable bacteria in a test food vehicle. And 1 log reduction would reduce the number of bacteria 90%. The 5 log refers to 10 to the 5th power or reduction in the number of microorganisms by 100,000-fold. For example, a product containing 100,000 pertinent microorganisms, a 5-log reduction would reduce the number or pertinent micro-organisms by 99.999%.

Two Practical Ways of Looking at 5-Log Reduction:
1. Reduction of 100,000 bad microorganisms in one contaminated serving to 1 bad microorganism in a serving.
2. Reduction of 100,000 contaminated servings to 1 contaminated serving.

Log Reduction Chart

| | % Reduction of |
|---|---|
| Log Reduction | Bacteria |
| 1 | 90 |
| 2 | 99 |
| 3 | 99.9 |
| 4 | 99.99 |
| 5 | 99.999 |

The surface tension of a liquid is the attractive force exerted by the molecules below the surface upon those at the surface/air interface. An internal pressure is thus created, which tends to restrain the liquid from flowing. Water is typically around 72 dynes/cm at 20° C.

The viscosity is the internal resistance to flow exhibited by a fluid, the ratio of shearing stress to rate of shear. The unit of viscosity is poise which equals 100 centipoise.

The fragrance test consists of subjectively determining the pleasantness of the odor of the moistening fluid by a panel of individuals.

Turbidity is a measurement of the cloudiness of a liquid caused by the presence of finely divided suspended material in the liquid. The Hach Ratio TM/XR Turbidimeter is a laboratory nephelometer capable of measuring

Turbidities up to 1999 nephelometric turbidity units (NTU) and as low as 0.001 NTU. The manufacturer of the above Turbidimeter is the Hach Company, which is located at P.O. Box 389, Loveland, Colo. 80539.

Procedure: A beam of light is directed through the test sample. Detectors are placed to measure the 90-degree light scatter, the forward scattered light and the light transmitted through the sample. The particles in the path of the light cause the NTU readings to result in increased values.

To determine the amount of time rolling tack which will be produced by an evaporated moistening fluid film on a Rolling Ramp Tack Tester is described below.

Procedure: The moistening fluid for each example was formulated and mixed by a magnetic stir bar. Approximately 300 ml of fluid was transferred to a glass wide mouth jar. The jar without a top was placed into an oven at 60 degrees Celsius for at least 2.5 days. The jar was removed from the oven and allowed to cool to ambient condition (15 minutes). The open jar was weighed on a Mettler Balance to the nearest 1/100 of a gram. The weight was recorded and then the jar was placed into the oven for another hour. The jar was taken out of the oven allowed to cool again and weighed. If the weight was similar to the first value the low boiling residuals were removed from the fluid. This sample will be used for coatings on plastic film. This accelerated evaporation is similar to the residues remaining on surfaces after the moistening fluid is allowed to evaporate. If the weight was less than the original value the jar was placed again into the oven until the weight stabilized. The remaining residual in the jar will be applied to a thin 0.01 mm clear polyester film.

The film was cut into a section 30 cm long by 20 cm wide and placed onto a K Control Coater 101, manufactured by RK Print Coat Instruments Limited, Litlington, Royston, Herts, SG8 OQZ, United Kingdom, to apply the moistening fluid residual. The K Control Coater is a metal flat bed with a thin layer of elastomer sheet and plastic sheet. The K Coater allows for the attachment of K Lox flexi graphic proofing kit consisting of a metal engraved anilox 200 cell/per linear inch roller with cell volume of 10.2 cubic centimeters per square meter and rubber roller all manufactured by RK Print Coat Instruments LTD.

The coating machine base unit has an on/off switch and speed control and forward and reverse control. A sheet of spoil paper was placed on the base coating machine and held in place with a clip to absorb excessive residue. The clear polyester previously cut sheet was placed on the spoil paper and held with a clip. Next the K Lox unit was secured to the base unit by tightening the knobs at each end. A spatula was used to remove residue from the sample jar. The residue was poured onto the full length of rubber roller nip to metal roller. The speed of the coating unit was set at 18.3 cm per second. The coated sheet was allowed to set at ambient for 15 to 30 minutes. The coated sheet was cut into 6.0 cm wide sections of the clear polyester substrate. This sheet was placed coated side up onto the Rolling Ramp Tack Tester.

The Rolling Ramp Tack Tester consists of an aluminum sheet with dimensions of length 55.5 cm, width 9 cm and thickness 0.37 cm. One side of the ramp is sitting on a laboratory jack. The height of the jack is 4.66 cm and thickness of ramp is 0.37 cm making a total upright height of 5.03 cm. The other end of the ramp is sitting on a flat surface. This creates the incline for the 100 gram brass roller with length 3.45 cm and diameter of 2.21 cm. On one edge of the ramp are two Omicron sensors (325 Mb EE-SPY 302) 23 cm apart. The sensors are connected to a power supply providing 5 volts which is also connected to an oscilloscope (Tektronic TDS 3034B 4-channel with digital color) to display the output. The sensors read the time it take for the roller to travel the 23 cm distance. A section of uncoated film was cut to length 30 cm and 6.5 cm wide and placed on the ramp. The roller was placed <1.0 cm away from the first sensor at the highest angle of the ramp and released, to roll down the ramp and pass the second sensor. The oscilloscope provided the time for the roller to travel to the second sensor a distance of 23 cm. This was repeated five times and the milliseconds recorded. The uncoated film was removed from the ramp. A coated sheet was placed on the Rolling Ramp Tack Tester. The roller was cleaned with acetone dried and placed <1.0 cm from the first sensor then released. The time was recorded and the roller was cleaned again with acetone. The sheet was removed from the ramp. A new coated sheet was placed on the ramp and previous steps repeated. A total of five-coated sections were tested and average time and standard deviation recorded.

Test Results for the Rolling Ramp Tack Tester when using no moistening fluid and uncoated film.
The rolling tack in milliseconds first try is 768.
The rolling tack in milliseconds second try is 768.
The rolling tack in milliseconds third try is 768.
The rolling tack in milliseconds fourth try is 768.
The rolling tack in milliseconds fifth try is 768.
The average rolling tack in milliseconds is 768.
The standard deviation of the rolling tack in milliseconds is 0.
The following examples are exemplary of the invention and should not be considered as limiting.

### EXAMPLE 1

Composition

| | | |
|---|---|---|
| Fragrance | French Vanilla 0.035 | 0.035 wt % |
| Detergent | X-20146 | 5.0 wt % |
| Biocide | Alkyl (C₁₄ 50%; C₁₆ 10%, C₁₂ 40%) | 0.023 wt % |
| | Dimethyl Benzyl Ammonium | |
| | Chloride | |
| Alcohol | 2-Propanol | 4.950 wt % |
| Dye | FD &C Blue#1 | 0.00015 wt % |
| De-ionized | | 89.99199 wt % |
| Water | | |

Properties of Example 1
The rolling tack in milliseconds first try is 908.
The rolling tack in milliseconds second try is 1,560.
The rolling tack in milliseconds third try is 1,760.
The rolling tack in milliseconds fourth try is 1,100.
The rolling tack in milliseconds fifth try is 1,100.
The average rolling tack in milliseconds is 1,285.
The standard deviation of the rolling tack in milliseconds is 395.
The viscosity is 1.14 centipose.
The surface tension of the moistening fluid is 40.6 dynes/cm.
The pH of the moistening fluid is 5.26.
The brush wick rate is 2.85 seconds.
The turbidity on a NTU 2 scale is 1.973.
The envelope sealing in grams is 2.83.
The conductivity of the moistening fluid is 54 micromho.
The specific gravity is 0.9937.
The Zone of inhibition is 4.0 mm with a 16 mm saturated disc at 24 hours.
The Zone of inhibition is 4.0 mm with a 16 mm saturated disc at 48 hours.
The Zone of inhibition is 4.0 mm with a 16 mm saturated disc at 72 hours.
The moistening fluid had an acceptable odor.

Wicking Test Times:

| | |
|---|---|
| Measurement No. 1: | 0.75 seconds |
| Measurement No. 2: | 1.00 seconds |
| Measurement No. 3: | 0.95 seconds |
| Average Measurement: | 0.90 seconds |

Performance of Example 1
1. The moistening fluid's biocidal capability was at an acceptable level with no regrowth visible in the zone of inhibition.
2. The results of the sealing test were satisfactory, in that all the envelopes were sealed.
3. The challenge test results indicated acceptable immediate and sustained microbial log reduction of target organisms.
4. The viscosity, surface tension, turbidity and pH were within an acceptable range.

### EXAMPLE 2

Composition

| | | |
|---|---|---|
| Fragrance | French Vanilla | 0.035 wt % |
| Detergent | X-20146 | 0.5 wt % |
| Biocide | Alkyl (C14 50%; C16 10%, C12 | 0.023 wt % |
| | 40%) Dimethyl Benzyl | |
| | Ammonium Chloride | |
| Alcohol | 2-Propanol | 4.95 wt % |
| Dye | FD &C Blue#1 | 0.000015 wt % |
| De-ionized Water | | 94.28499 wt % |

Properties of Example 2
The rolling tack in milliseconds first try is 932.
The rolling tack in milliseconds second try is 1,620.
The rolling tack in milliseconds third try is 1,600.
The rolling tack in milliseconds fourth try is 1,600.
The rolling tack in milliseconds fifth try is 1,590.
The average rolling tack in milliseconds is 1, 438.
The standard deviation of the rolling tack in milliseconds is 337.
The viscosity is 1.03 centipose.
The surface tension of the moistening fluid is 39.2 dynes/cm.
The pH of the moistening fluid is 5.26.
The brush wick rate is 0.91 seconds.
The turbidity on a NTU 2 scale is 0.541.
The envelope sealing in grams is 2.63.
The conductivity of the moistening fluid is 41.3 micromho.
The specific gravity is 0.9922.
The Zone of Inhibition is 3.0 mm with a 16 mm saturated disc at 24 hours.
The Zone of Inhibition is 3.0 mm with a 16 mm saturated disc at 48 hours.
The Zone of Inhibition is 3.0 mm with a 16 mm saturated disc at 72 hours.
The moistening fluid had an acceptable odor.

Wicking Test Times

| | |
|---|---|
| Measurement No. 1: | 1.79 seconds |
| Measurement No. 2: | 1.50 seconds |
| Measurement No. 3: | 1.03 seconds |
| Average Measurement: | 1.44 seconds |

Performance of Example 2
1. The moistening fluid's biocidal capability was at an acceptable level with no regrowth visible in the zone of inhibition.
2. The results of the sealing test were satisfactory, in that all the envelopes sealed.
3. The viscosity, surface tension, turbidity and pH were within an acceptable range.

### EXAMPLE 3

Composition

| | | |
|---|---|---|
| Fragrance | French Vanilla | 0.035 wt % |
| Detergent | X-20146 | 0.25 wt % |
| Biocide | Alkyl (C14 50%; C16 10%, C12 | 0.023 wt % |
| | 40%) Dimethyl Benzyl | |
| | Ammonium Chloride | |
| Alcohol | 2-Propanol | 4.95 wt % |
| Dye | FD &C Blue#1 | 0.000015 wt % |
| De-ionized Water | | 94.76499 wt % |

Properties of Example 3
The rolling tack in milliseconds first try is 1040.
The rolling tack in milliseconds second try is 996.
The rolling tack in milliseconds third try is 1,290.
The rolling tack in milliseconds fourth try is 1,450.
The rolling tack in milliseconds fifth try is 1,620.
The average rolling tack in milliseconds is 1,194.
The standard deviation of the rolling tack in milliseconds is 214.
The viscosity is 1.01 centipose.
The surface tension of the moistening fluid is 35.6 dynes/cm.
The pH of the moistening fluid is 4.88.
The brush wick rate is 2.34 seconds.
The turbidity on a NTU 2 scale is 0.392.
The envelope sealing in grams is 3.96 g.
The conductivity of the moistening fluid is 37.9 micromho.
The specific gravity is 0.9922.
The Zone of Inhibition is 2.0 mm with a 16 mm saturated disc at 24 hours.
The Zone of Inhibition is 2.0 mm with a 16 mm saturated disc at 48 hours.
The Zone of Inhibition is 2.0 mm with a 16 mm saturated disc at 72 hours.
The moistening fluid had an acceptable odor.

Wicking Test Time - 2.96 secs.

| | |
|---|---|
| Measurement No. 1: | 1.10 seconds |
| Measurement No. 2: | 0.74 seconds |
| Measurement No. 3: | 0.60 seconds |
| Average Measurement: | 0.81 seconds |

Performance of Example 3:
1. The moistening fluid's biocidal capability was at an acceptable level with no regrowth visible in the zone of inhibition.
2. The results of the sealing test were satisfactory, in that all the envelopes sealed.
3. The viscosity, surface tension, turbidity and pH were within an acceptable range.
4. The moistening fluid's biocidal capability was at an acceptable level with no regrowth visible in the zone of inhibition.

### EXAMPLE 4

Composition

| | | |
|---|---|---|
| Fragrance | Peppermint | 0.035 wt % |
| Detergent | Burco LAF-3420 | 5.0 wt % |
| Biocide | Alkyl (C₁₄ 50%; C₁₆ 10%, C₁₂ 40%) | 0.023 wt % |
| | Dimethyl Benzyl Ammonium | |
| | Chloride | |
| Alcohol | 2-Propanol | 4.95 wt % |
| Dye | FD &C Blue#1 | 0.000015 wt % |
| De-ionized Water | | 89.99199 wt % |

Properties of Example 4
The rolling tack in milliseconds first try is 968.
The rolling tack in milliseconds second try is 1,000.
The rolling tack in milliseconds third try is 940.
The rolling tack in milliseconds is fourth try 1,110.
The rolling tack in milliseconds fifth try is 1,770.
The average rolling tack in milliseconds is 1,004.
The standard deviation of the rolling tack in milliseconds is 74.
The viscosity is 1.19 centipose.
The surface tension of the moistening fluid is 33.7 dynes/cm.
The pH of the moistening fluid is 3.81.
The brush wick rate is 1.02 seconds.
The turbidity on a NTU 20 scale is 2.08.
The envelope sealing in grams is 3.77 g.
The conductivity of the moistening fluid is 70.1 micromho.
The specific gravity is 0.994.
The Zone of Inhibition is 5.0 mm with a 16 mm saturated disc at 24 hours.
The Zone of Inhibition is 5.0 mm with a 16 mm saturated disc at 48 hours.
The Zone of Inhibition is 5.0 mm with a 16 mm saturated disc at 72 hours.
The moistening fluid had an acceptable odor.

Wicking Test Times

| | |
|---|---|
| Measurement No. 1: | 1.20 seconds |
| Measurement No. 2: | 1.60 seconds |
| Measurement No. 3: | 1.59 seconds |
| Average Measurement: | 1.46 seconds |

Performance of Example 4.
1. The moistening fluid's biocidal capability was at an acceptable level with no regrowth visible in the zone of inhibition.
2. The results of the sealing test were satisfactory, in that all the envelopes sealed.
3. The viscosity, surface tension, turbidity and pH were within an acceptable range.

### EXAMPLE 5

Composition

| | | |
|---|---|---|
| Fragrance | Peppermint A44681 | 0.035 wt % |
| Detergent | Burco LAF-3420 | 0.50 wt % |
| Biocide | Alkyl (C₁₄ 50%; C₁₆ 10%, C₁₂ 40%) | 0.023 wt % |
| | Dimethyl Benzyl Ammonium | |
| | Chloride | |
| Alcohol | 2-Propanol | 4.95 wt % |
| Dye | FD &C Blue#1 | 0.000015 wt % |
| De-ionized Water | | 94.49199 wt % |

Properties of Example 5
The rolling tack in milliseconds first try is 872.
The rolling tack in milliseconds second try is 1,120.
The rolling tack in milliseconds third try is 924.
The rolling tack in milliseconds fourth try is 1,630.
The rolling tack in milliseconds fifth try is 1,680.
The average rolling tack in milliseconds is 1,161.
The standard deviation of the rolling tack in milliseconds is 347.
The viscosity is 1.07 centipose.
The surface tension of the moistening fluid is 34.7 dynes/cm.
The pH of the moistening fluid is 4.33.
The brush wick rate is 2.76 seconds.
The turbidity on a NTU 2 scale is 0.351.
The envelope sealing in grams is 2.49 g.
The conductivity of the moistening fluid is 45.5 micromho.
The specific gravity is 0.992.
The Zone of Inhibition is 2.5 mm with a 16 mm saturated disc at 24 hours.
The Zone of Inhibition is 2.5 mm with a 16 mm saturated disc at 48 hours.
The Zone of Inhibition is 2.5 mm with a 16 mm saturated disc at 72 hours.
The moistening fluid had an acceptable odor.

Wicking Test Times

| | |
|---|---|
| Measurement No. 1: | 0.91 seconds |
| Measurement No. 2: | 1.20 seconds |
| Measurement No. 3: | 0.90 seconds |
| Average Measurement: | 1.00 seconds |

Performance of Example 5
1. The moistening fluid's biocidal capability was at an acceptable level with no regrowth visible in the zone of inhibition.
2. The results of the sealing test were satisfactory, in that all the envelopes sealed.
3. The viscosity, surface tension, turbidity and pH were within an acceptable range.

### EXAMPLE 6

Composition

| | | |
|---|---|---|
| Fragrance | Peppermint A44681 | 0.035 wt % |
| Detergent | Burco LAF-3420 | 0.25 wt % |
| Biocide | Alkyl (C₁₄ 50%; C₁₆ 10%, C₁₂ 40%) | 0.023 wt % |
| | Dimethyl Benzyl Ammonium | |
| | Chloride | |
| Alcohol | 2-Propanol | 4.95 wt % |
| Dye | FD &C Blue#1 | 0.000015 wt % |
| De-ionized Water | | 94.74199 wt % |

Properties of Example 6
The rolling tack in milliseconds first try is 900.
The rolling tack in milliseconds second try is 1,530.
The rolling tack in milliseconds third try is 1,220.
The rolling tack in milliseconds fourth try is 1,180.
The rolling tack in milliseconds fifth try is 980.
The average rolling tack in milliseconds is 1,207.
The standard deviation of the rolling tack in milliseconds is 257.
The viscosity is 1.03 centipose.
The surface tension of the moistening fluid is 35.4 dynes/cm.
The pH of the moistening fluid is 4.48.
The brush wick rate is 1.85 seconds.
The turbidity on a NTU 2 scale is 0.168.
The sealing in grams is 2.49.
The conductivity of the moistening fluid is 41.3 micromho.
The specific gravity is 0.9919.
The Zone of Inhibition is 4.75 mm with a 45 mm saturated disc at 48 hours.
The Zone of Inhibition is 5.0 mm with a 45 mm saturated disc at 72 hours.
The moistening fluid had an acceptable odor.

Wicking Test Times

| | |
|---|---|
| Measurement No. 1: | 0.35 seconds |
| Measurement No. 2: | 0.65 seconds |
| Measurement No. 3: | 0.95 seconds |
| Average Measurement: | 0.65 seconds |

Performance of Example 6
1. The moistening fluid's biocidal capability was at an acceptable level.
2. The results of the sealing test were satisfactory, in that all the envelopes sealed.
3. The viscosity, surface tension, turbidity and pH were within an acceptable range.

### EXAMPLE 7

Composition

| | | | |
|---|---|---|---|
| Fragrance | Citrus 6001578 | 0.035 | wt % |
| Detergent | Burco LAF-345 PB | 5.0 | wt % |
| Biocide | Alkyl (C₁₄ 50%; C₁₆ 10%, C₁₂ 40%) | 0.023 | wt % |
| | Dimethyl Benzyl Ammonium Chloride | | |
| Alcohol | 2-Propanol | 4.95 | wt % |
| Dye | FD &C Blue#1 | 0.000015 | wt % |
| De-ionized | | 89.99199 | wt % |
| Water | | | |

Properties of Example 7
The rolling tack in milliseconds first try is 828.
The rolling tack in milliseconds second try is 856.
The rolling tack in milliseconds third try is 1,060.
The rolling tack in milliseconds fourth try is 856.
The rolling tack in milliseconds fifth try is 936.
The average rolling tack in milliseconds is 907.
The standard deviation of the rolling tack in milliseconds is 95.
The viscosity is 1.46 centipose.
The surface tension of the moistening fluid is 31.1 dynes/cm.
The pH of the moistening fluid is 6.01.
The brush wick rate is 2.56 seconds.
The turbidity on a NTU 20 scale is 2.43.
The sealing in grams is 2.89.
The conductivity of the moistening fluid is 93.5 micromho.
The specific gravity is 0.9942.
The Zone of Inhibition is 5.00 mm with a 45 mm saturated disc at 24 hours.
The Zone of Inhibition is 5.00 mm with a 45 mm saturated disc at 48 hours.
The Zone of Inhibition is 5.00 mm with a 45 mm saturated disc at 72 hours.
The moistening fluid had an acceptable odor.

Wicking Test Times

| | |
|---|---|
| Measurement No. 1: | 0.70 seconds |
| Measurement No. 2: | 0.75 seconds |
| Measurement No. 3: | 0.80 seconds |
| Average Measurement: | 0.75 seconds |

Performance of Example 7
1. The moistening fluid's biocidal capability was acceptable at an acceptable level.
2. The results of the sealing test were satisfactory, in that all the envelopes sealed.
3. The viscosity, surface tension, turbidity and pH were within an acceptable range.

### EXAMPLE 8

Composition

| | | | |
|---|---|---|---|
| Fragrance | .035 Citrus 6001578 | 0.035 | wt % |
| Detergent | Burco LAF-345 PB | 0.50 | wt % |
| Biocide | Alkyl (C₁₄ 50%; C₁₆ 10%, C₁₂ 40%) | 0.023 | wt % |
| | Dimethyl Benzyl Ammonium Chloride | | |
| Alcohol | 2-Propanol | 4.95 | wt % |
| Dye | FD &C Blue#1 | 0.000015 | wt % |
| De-ionized | | 94.49199 | wt % |
| Water | | | |

Properties of Example 8
The rolling tack in milliseconds first try is 832.
The rolling tack in milliseconds second try is 940.
The rolling tack in milliseconds third try is 852.
The rolling tack in milliseconds fourth try is 884.
The rolling tack in milliseconds fifth try is 812.
The average rolling tack in milliseconds is 877.
The standard deviation of the rolling tack in milliseconds is 47.
The viscosity is 1.03 centipose.
The surface tension of the moistening fluid is 29.8 dynes/cm.
The pH of the moistening fluid is 5.31.
The brush wick rate is. 2.7 seconds.
The turbidity on a NTU 2 scale is 0.453.
The sealing in grams is 3.85.
The conductivity of the moistening fluid is 36.9 micromho.
The specific gravity is 0.992.
The Zone of Inhibition is 5.00 mm with a 45 mm saturated disc at 24 hours.
The Zone of Inhibition is 5.00 mm with a 45 mm saturated disc at 48 hours.
The Zone of Inhibition is 5.00 mm with a 45 mm saturated disc at 72 hours.
The moistening fluid had an acceptable odor.

Wicking Test Times

| | |
|---|---|
| Measurement No. 1: | 0.84 seconds |
| Measurement No. 2: | 0.85 seconds |
| Measurement No. 3: | 0.98 seconds |
| Average Measurement: | 0.89 seconds |

Challenge Test

Log Reductions

### Results:

| Organism | 24 Hours | 48 Hours | 72 Hours | 7 Days |
|---|---|---|---|---|
| Pseudomonas aeruginosa | 2 | 3 | 4 | 5 |
| Cladosporium | 2 | 3 | 3 | 4 |
| cladosporioides | | | | |
| Geotrichum candidum | 2 | 3 | 3 | 4 |

Performance of Example 8
1. The moistening fluid's biocidal capability was at an acceptable level.
2. The results of the sealing test were satisfactory, in that all the envelopes sealed.
3. The viscosity, surface tension, turbidity and pH were within an acceptable range.

### EXAMPLE 9

Composition

| | | | |
|---|---|---|---|
| Fragrance | Citrus 6001578 | 0.035 | wt % |
| Detergent | Burco LAF-345 PB | 0.25 | wt % |
| Biocide | Alkyl (C₁₄ 50%; C₁₆ 10%, C₁₂ 40%) | 0.023 | wt % |
| | Dimethyl Benzyl Ammonium Chloride | | |
| Alcohol | 2-Propanol | 4.95 | wt % |
| Dye | FD &C Blue#1 | 0.000015 | wt % |
| De-ionized | | 94.74199 | wt % |
| Water | | | |

Properties of Example 9
The rolling tack in milliseconds first try is 876.
The rolling tack in milliseconds second try is 888.
The rolling tack in milliseconds third try is 800.
The rolling tack in milliseconds fourth try is 904.
The rolling tack in milliseconds fifth try is 812.
The average rolling tack in milliseconds is 867.
The standard deviation of the rolling tack in milliseconds is 46.
The viscosity is 1.08 centipose.
The surface tension of the moistening fluid is 27.9 dynes/cm.
The pH of the moistening fluid is 5.39.
The brush wick rate is 2.17 seconds.
The turbidity on a NTU 20 scale is 1.98.
The sealing in grams is 3.81.
The conductivity of the moistening fluid is 21.3 micromho.
The specific gravity is 0.9917.
The Zone of Inhibition is 6.0 mm with a 45 mm saturated disc at 24 hours.
The Zone of Inhibition is 5.0 mm with a 45 mm saturated disc at 48 hours.
The Zone of Inhibition is 4.0 mm with 45 mm saturated disc at 72 hours.
The moistening fluid had an acceptable odor.

Wicking Test Times

| | |
|---|---|
| Measurement No. 1: | 1.00 seconds |
| Measurement No. 2: | 0.95 seconds |
| Measurement No. 3: | 1.20 seconds |
| Average Measurement: | 1.05 seconds |

Performance of Example 9
1. The moistening fluid's biocidal capability was at an acceptable level with no regrowth visible in the zone of inhibition.
2. The results of the sealing test were satisfactory, in that all the envelopes sealed.
3. The viscosity, surface tension, turbidity and pH were within an acceptable range.

### EXAMPLE 10

Composition

| | | | |
|---|---|---|---|
| Fragrance | .035 Citrus 6001578 | 0.035 | wt % |
| Detergent | Burco HCS-50NF | 5.0 | wt % |
| Biocide | Alkyl (C₁₄ 50%; C₁₆ 10%, C₁₂ 40%) | 0.023 | wt % |
| | Dimethyl Benzyl Ammonium Chloride | | |
| Alcohol | 2-Propanol | 4.95 | wt % |
| Dye | FD &C Blue#1 | 0.000015 | wt % |
| De-ionized | | 89.99199 | wt % |
| Water | | | |

Properties of Example 10
The rolling tack in milliseconds first try is 872.
The rolling tack in milliseconds second try is 996.
The rolling tack in milliseconds third try is 1,300.
The rolling tack in milliseconds fourth try is 1,410.
The rolling tack in milliseconds fifth try is 1,460.
The average rolling tack in milliseconds is 1,114.
The standard deviation of the rolling tack in milliseconds is 252.
The viscosity is 1.07 centipose.
The surface tension of the moistening fluid is 27.7 dynes/cm.
The pH of the moistening fluid is 5.38.
The brush wick rate is 1.85 seconds.
The turbidity on a NTU 2 scale is 0.63.
The sealing in grams is 3.75.
The conductivity of the moistening fluid is 2.61 millimhos.
The specific gravity is 0.9997.
The Zone of Inhibition is 5.0 mm with a 16 mm saturated disc at 24 hours.
The Zone of Inhibition is 5.0 mm with a 16 mm saturated disc at 48 hours.
The Zone of Inhibition is 5.0 mm with a 16 mm saturated disc at 72 hours.
The moistening fluid had an acceptable odor.

Wicking Test Time—
Measurement No. 1: 1.10 seconds
Measurement No. 2: 0.74 seconds
Measurement No. 3: 0.60 seconds
Average Measurement: 0.81 seconds

Challenge Test

Log Reductions

| Organism | Zero Time | 24 Hours | 48 Hours | 72 Hours | 7 Days |
|---|---|---|---|---|---|
| Caulobacter | >3.7 | >3.7 | >3.7 | >3.7 | >4.7 |
| Acinetobacter | >3.8 | >3.8 | >3.8 | >3.8 | >4.8 |
| Penicillium | >3.5 | >3.5 | >3.5 | >3.5 | >4.5 |
| Cladosporium | >3.7 | >3.7 | >3.7 | >3.7 | >4.7 |
| Geotrichum | >3.5 | >3.5 | >3.5 | >3.5 | >4.5 |
| Algae | — | >4.3 | >4.3 | >4.3 | >4.3 |
| E coli | >3.1 | >3.1 | >3.1 | >3.1 | >4.1 |
| Pseudomonas | >3.5 | >3.5 | >3.5 | >3.5 | >4.4 |
| Candida | 2.5 | >3.2 | >3.2 | >3.2 | >4.2 |
| Aspergillus | 0.96 | >3.0 | >3.0 | >3.0 | >4.0 |
| Sacchromyces | >3 | >3.0 | >3.0 | >3.0 | >4.0 |
| cereviseae | | | | | |

Performance of Example 10:
1. The moistening fluid's biocidal capability was at an acceptable level with no regrowth visible in the zone of inhibition.
2. The results of the sealing test were satisfactory, in that all the envelopes sealed.
3. The viscosity, surface tension, turbidity and pH were within an acceptable range.
4. The challenge test results indicated acceptable immediate and sustained microbial log reduction of target organisms.

### EXAMPLE 11

Composition

| | | | |
|---|---|---|---|
| Fragrance | Citrus 6001578 | 0.035 | wt % |
| Detergent | Burco HCS-50NF | 0.50 | wt % |
| Biocide | Alkyl (C₁₄ 50%; C₁₆ 10%, C₁₂ 40%) | 0.023 | wt % |
| | Dimethyl Benzyl Ammonium Chloride | | |
| Alcohol | 2-Propanol | 4.95 | wt % |
| Dye | FD &C Blue#1 | 0.000015 | wt % |
| De-ionized | | 94.49199 | wt % |
| Water | | | |

Properties of Example 11
The rolling tack in milliseconds first try is 916.
The rolling tack in milliseconds second try is 1,200.
The rolling tack in milliseconds third try is 1,040.
The rolling tack in milliseconds fourth try is 1,020.
The rolling tack in milliseconds fifth try is 1,070.
The average rolling tack in milliseconds is 1,044.
The standard deviation of the rolling tack in milliseconds is 117.
The viscosity is 0.97 centipose.
The surface tension of the moistening fluid is 33.0 dynes/cm.
The pH of the moistening fluid is 5.58.
The brush wick rate is 1.06 seconds.
The turbidity on a NTU 20 scale is 10.09.
The sealing in grams is 3.46.
The conductivity of the moistening fluid is 289 micromho.
The specific gravity is 0.9926.
The Zone of Inhibition is 6.0 mm with a 16 mm saturated disc at 24 hours.
The Zone of Inhibition is 6.0 mm with a 16 mm saturated disc at 48 hours.
The Zone of Inhibition is 6.0 mm with a 16 mm saturated disc at 72 hours.
The moistening fluid had an acceptable odor.

Wicking Test Times

| | |
|---|---|
| Measurement No. 1: | 0.91 seconds |
| Measurement No. 2: | 1.20 seconds |
| Measurement No. 3: | 0.90 seconds |
| Average Measurement: | 1.00 seconds |

Performance of Example 11
1. The moistening fluid's biocidal capability was at an acceptable level with no regrowth visible in the zone of inhibition.
2. The results of the sealing test were satisfactory, in that all the envelopes sealed.
3. The viscosity, surface tension, turbidity and pH were within an acceptable range.

### EXAMPLE 12

Composition

| | | | |
|---|---|---|---|
| Fragrance | Citrus 6001578 | 0.035 | wt % |
| Detergent | Burco HCS-50NF | 0.50 | wt % |
| Biocide | Alkyl (C₁₄ 50%; C₁₆ 10%, C₁₂ 40%) | 0.023 | wt % |
| | Dimethyl Benzyl Ammonium Chloride | | |
| Alcohol | 2-Propanol | 4.95 | wt % |
| Dye | FD &C Blue#1 | 0.000015 | wt % |
| De-ionized | | 94.49199 | wt % |
| Water | | | |

Properties of Example 12
The rolling tack in milliseconds first try is 864.
The rolling tack in milliseconds second try is 1,240.
The rolling tack in milliseconds third try is 856.
The rolling tack in milliseconds fourth try is 858.
The rolling tack in milliseconds fifth try is 832.
The average rolling tack in milliseconds is 954.
The standard deviation of the rolling tack in milliseconds is 190.
The viscosity is 1.01 centipose.
The surface tension of the moistening fluid is 27.1 dynes/cm.
The pH of the moistening fluid is 5.06.
The brush wick rate is 3.22 seconds.
The turbidity on a NTU 20 scale is 7.66.
The sealing in grams is 3.98.
The conductivity of the moistening fluid is 187.9 micromho.
The specific gravity is 0.9924.
The Zone of Inhibition is 6.0 mm with a 45 mm saturated disc at 24 hours.
The Zone of Inhibition is 6.0 mm with a 45 mm saturated disc at 48 hours.
The Zone of Inhibition is 6.0 mm with 45 mm saturated disc at 72 hours.
The moistening fluid had an acceptable odor.

Wicking Test Times

| | |
|---|---|
| Measurement No. 1: | 1.00 seconds |
| Measurement No. 2: | 0.95 seconds |
| Measurement No. 3: | 1.20 seconds |
| Average Measurement: | 1.05 seconds |

Performance of Example 12
1. The moistening fluid's biocidal capability was at an acceptable level with no regrowth visible in the zone of inhibition.
2. The results of the sealing test were satisfactory, in that all the envelopes sealed.
3. The viscosity, surface tension, turbidity and pH were within an acceptable range.

### EXAMPLE 13

### 100% Distilled Water

Properties of Example 13
The pH of the moistening fluid is 6.7.
The conductivity of the moistening fluid is 3.3 micromhos
The surface tension of this moistening fluid is 67.4 dynes/cm
The Zone of Inhibition is 0.0 mm with a 16 mm saturated disc at 24 hours.
The Zone of Inhibition is 0.0 mm with a 16 mm saturated disc at 48 hours.
The Zone of Inhibition is 0.0 mm with a 16 mm saturated disc at 72 hours.
The sealing in grams is 0.704.

Wicking Test Times

| | |
|---|---|
| Measurement No. 1: | 2.15 seconds |
| Measurement No. 2: | 1.43 seconds |
| Measurement No. 3: | 2.22 seconds |
| Average Measurement: | 1.93 seconds |

Viscosity - 0.89 cps

Performance of Example 13
1. The moistening fluid's biocidal capability was not acceptable, as there was no zone of inhibition.
2. The results of the sealing test were satisfactory, in that all the envelopes sealed.

### EXAMPLE 14

### 100% Deionized Water

Properties of Example 14
The pH of the moistening fluid is 6.7.
The conductivity of the moistening fluid is 1.1 micromohs.
The surface tension of this moistening fluid is 59.5 dynes/cm.
The Zone of Inhibition is 0.0 mm with a 16 mm saturated disc at 24 hours.
The Zone of Inhibition is 0.0 mm with a 16 mm saturated disc at 48 hours.
The Zone of Inhibition is 0.0 mm with a 16 mm saturated disc at 72 hours.
The sealing in grams is 0.532.

Wicking Test Times

| | |
|---|---|
| Measurement No. 1: | 1.61 seconds |
| Measurement No. 2: | 1.35 seconds |
| Measurement No. 3: | 1.84 seconds |
| Average Measurement: | 1.60 seconds |

Viscosity—0.89 cps.

Performance of Example 14
1. The moistening fluid's biocidal capability was not acceptable as there was no zone of inhibition.
2. The results of the sealing test were satisfactory, in that all the envelopes sealed.

### EXAMPLE 15

### 100% Tap Water

Properties of Example 15
The pH of the moistening fluid is 6.3.
The conductivity of the moistening fluid is 167.3 micromohs.
The surface tension of this moistening fluid is 58.1 dynes/cm.
The Zone of Inhibition is 0.0 mm with a 16 mm saturated disc at 24 hours.
The Zone of Inhibition is 0.0 mm with a 16 mm saturated disc at 48 hours.
The Zone of Inhibition is 0.0 mm with a 16 mm saturated disc at 72 hours.
The sealing in grams is 0.0 g.

Wicking Test Time

| | |
|---|---|
| Measurement No. 1: | 3.30 seconds |
| Measurement No. 2: | 3.56 seconds |
| Measurement No. 3: | 3.71 seconds |
| Average Measurement: | 3.52 seconds |

Viscosity—1.00 cps.

Challenge Test

Log Reductions

| Organism | Zero Time | 24 Hours | 48 Hours | 72 Hours | 7 Days |
|---|---|---|---|---|---|
| Caulobacter | 0 | 0 | 0 | 0 | 0 |
| Pseudomonas | 0 | 0 | 0 | 0 | 0 |
| Candida | 0 | 0 | 0 | 0 | 0 |
| Aspergillus | 0 | 0 | 0 | 0 | 0 |
| Sacchromyces | 0 | 0 | 0 | 0 | 0 |
| cereviseae | | | | | |

Performance of Example 15
1. The moistening fluid's biocidal capability was not acceptable as there was no zone of inhibition.
2. The results of the sealing test were satisfactory, in that all the envelopes sealed.
3. The challenge test results indicated no log reduction of target organisms and therefore were unacceptable.

The above embodiments have been given by way of illustration only, and other embodiments of the instant invention will be apparent to those skilled in the art, from consideration of the detailed description. Accordingly, any limitation on the instant invention is to be found only in the claims.

What is claimed is:

1. A moistening fluid consisting essentially of: detergent, biocide, alcohol, water and a fragrance; wherein the fluid inhibits and/or destroys the growth of specific bacteria, fungi and algae, while preventing a tacky residue build up upon the evaporation of the moistening fluid.

2. The fluid claimed in claim 1, wherein the fluid composition consists essentially of:
0.25-5.0 (wt %) Detergent;
0.023 (wt %) Biocide;
4.95 (wt %) Alcohol;
0.00015 (wt %) Dye;
0.035 (wt %) Fragrance; and
89.99199-94.74199 (wt %) Water.

3. The composition claimed in claim 2, wherein the detergents are selected from the group consisting of: Burco HCS-50NF, Burco LAF-3420, Burco LAF-345 PB and NF, X-20146

4. The composition claimed in claim 2, wherein the fragrances are selected from the group consisting of: Citrus 60001578 Scent, French Vanilla A44676 Scent, Pine Tree A44680 Scent and Peppermint A44681.

5. The composition claimed in claim 2, wherein the Biocide is Alkyl (C₁₄ 50%; C₁₆ 10%, C₁₂40%) Dimethyl Benzyl Ammonium Chloride.

6. The composition claimed in claim 2, wherein the Alcohol is 2-Propanol.

7. The composition claimed in claim 2, wherein the dye is Food Drug & Cosmetic #1 Blue Dye.

8. The fluid claimed in claim 2, wherein the pH of the fluid is between 3.81 and 6.01.

9. The fluid claimed in claim 2, wherein the surface tension of the fluid is between 27.1 dynes/cm and 40.6 dynes/cm.

10. The fluid claimed in claim 2, wherein the viscosity of the fluid is between 0.97 cps. and 1.46 cps.

11. The fluid claimed in claim 2, wherein the rolling track of the fluid is between 867 milliseconds and 1,438 milliseconds.

12. The fluid claimed in claim 2, wherein the brush wick rate of the fluid is between 1.02 seconds and 3.22 seconds.

13. The fluid claimed in claim 2, wherein the turbidity of the fluid is between 0.168 on a NTU 2 scale and 10.09 on a NTU 20 scale.

14. The fluid claimed in claim 2, wherein the envelope sealing of the fluid is between 2.49 grams and 3.98 grams.

15. The fluid claimed in claim 2, wherein the specific gravity of the fluid is between 0.9917 and 0.9997.

16. The fluid claimed in claim 1, wherein the fluid may be used to seal water moistening adhesive envelopes.

17. The fluid claimed in claim 1, wherein the fluid will inhibit and/or kill:
A. Sphingomonas paucimobilis (bacteria),
B. Pseudomonas aeruginosa (bacteria),
C. Pseudomonas stutzeri (bacteria),
D. Acinetobacter species (bacteria),
E. Caulobacter species (bacteria),
F. Pseudomonas fluorescens (bacteria),
G. Brevendimonas species (bacteria),
H. Flavomonas species (bacteria), and
I. Escherichia coli (bacteria).

18. The fluid claimed in claim 1, wherein the fluid will inhibit and/or kill:
A. Geotrichum species (fungus)
B. Cladosporium species (fungus), and
C. Oididendron species (fungus).

19. The fluid claimed in claim 1, wherein the fluid will inhibit and/or kill: yeasts.

20. The fluid claimed in claim 1, wherein the fluid may be will inhibit and/or kill:
Fusarium species (mold),
Aspergillus niger (mold); and
Penicillium species (mold).

21. The fluid claimed in claim 1, wherein the fluid will inhibit and/or kill:
Blue Green algae,
Freshwater algae.

22. The fluid claimed in claim 1, wherein the fluid may be used in a mailing system.

23. The fluid claimed in claim 1, wherein the fluid may be used in a moistening device.

24. The fluid claimed in claim 1, wherein the moistening device is moistening dispenser.

25. The moistening fluid claimed in claim 1, wherein the fluid further includes a dye.

26. The fluid claimed in claim 1, wherein the fluid may be used to adhere tapes to surfaces.

27. The fluid claimed in claim 1, wherein the fragrance masks the alcohol aroma.

28. A fragranced moistening fluid that inhibits and/or destroys the growth of specific bacteria, fungi and algae, while preventing a tacky residue build up upon the evaporation of the moistening fluid, consisting essentially of:
0.25-5.0 (wt %) Detergent;
0.023 (wt %) Biocide;
4.95 (wt %) Alcohol;
0.00015 (wt %) Dye;
0.035 (wt %) Fragrance; and
89.99199-94.74199 (wt %) Water.
